(19) United States
(12) Patent Application Publication
(10) Pub. No.: US 2025/0072797 A1
(43) Pub. Date: Mar. 6, 2025

(54) MICRONEEDLE SENSOR, METHOD OF MANUFACTURING THE SAME, AND CONTINUOUS BODY FLUID METER USING THE SAME

(71) Applicant: QuadMedicine, Inc., Seongnam-si (KR)

(72) Inventors: Seung Ki BAEK, Seoul (KR); Seong O CHOI, Namyangju-si (KR); Hye Seung SEON WOO, Seongnam-si (KR); Dae Kyung KIM, Seongnam-si (KR); Bo Yeong PARK, Gwangju-si (KR)

(73) Assignee: QuadMedicine, Inc., Seongnam-si (KR)

(21) Appl. No.: 18/824,417

(22) Filed: Sep. 4, 2024

(30) Foreign Application Priority Data
Sep. 5, 2023 (KR) 10-2023-0117664

(51) Int. Cl. A61B 5/1486; A61B 5/145

(52) U.S. Cl. CPC A61B 5/14865; A61B 5/14514; A61B 5/14532

(57) ABSTRACT

The microneedle sensor according to an embodiment of the present disclosure includes a sensor panel placed so that one surface thereof is in contact with the skin of the body; a plurality of hollow microneedles that are formed on one surface of the sensor panel to protrude in the form of micrometer-scale needles, wherein a hollow hole with one side exposed to the outside is provided inside the hollow microneedle; reaction electrodes of a conductive material each inserted into the hollow holes of the hollow microneedles and configured to be inserted into the interior of the skin along with the hollow microneedles; a reference electrode of a conductive material applied to a first region formed on one surface of the sensor panel and applied to the outer surfaces of hollow microneedles located on the first region; and a counter electrode of a conductive material applied to a second region formed on one surface of the sensor panel and applied to the outer surfaces of hollow microneedles located on the second region.

### CROSS-REFERENCE TO RELATED APPLICATION

This application claims priority to Korean Patent Application No. 10-2023-0117664, filed on Sep. 5, 2023, in the Korean Intellectual Property Office, the disclosure of which is incorporated herein by reference.

### BACKGROUND OF THE DISCLOSURE

### Field of the Disclosure

The present disclosure relates to a microneedle sensor, a method of manufacturing the same, and a continuous body fluid meter using the same. More particularly, the present disclosure relates to a microneedle sensor capable of continuously sensing body fluid components in a minimally invasive manner using hollow microneedles, increasing the placement density of reaction electrodes by improving the installation structure of the reaction electrodes (working electrodes), a counter electrode, or a reference electrode, and easily adjusting the sensing sensitivity of the reaction electrodes for specific substances in body fluid, a method of manufacturing the microneedle sensor, and a continuous body fluid meter using the microneedle sensor.

### Description of the Related Art

In general, a microneedle is a microneedle structure used to pierce the stratum corneum of the body and deliver drugs to the dermal layer, and is often manufactured in the form of a patch attached to the skin.

For example, a solid microneedle is used to create a microscopic hole in the skin and then apply a medicine onto the skin, a coated microneedle is used by applying a chemical to the surface of the needle and inserting the microneedle into the skin, a melting microneedle is used by inserting a microneedle containing a medicine into the skin and melting the medicine, and a hollow microneedle is used by piercing the skin and then injecting a medicine through a hollow hole.

Recently, technology has been developed to examine or diagnose body fluid components inside the skin using a microneedle. In particular, a biosensor has been developed to measure specific substances from body fluid such as blood or interstitial fluid (ISF) using a hollow microneedle.

For example, related art documents include Korean Patent No. 10-1542549 (Invention title: MICRONEEDLE ARRAY FOR BIOSENSING AND DRUG DELIVERY, registration date: Jul. 31, 2015) and Korean Patent Application Publication No. 10-2021-0127757 (Invention title: NEEDLE-TYPE BIOSENSOR, publication date: Oct. 22, 2021).

However, in Korean Patent No. 10-1542549, since any one of a reaction electrodes, a counter electrode, and a reference electrode must be placed in the hollow hole of a hollow microneedle, to use all reaction electrodes, counter electrode, and reference electrode, three or more hollow microneedles must be used. Thus, there is a problem of having to form multiple microneedles to ensure sensing range and accuracy.

In addition, in Korean Patent Application Publication No. 10-2021-0127757, according to a method of extracting and sampling body fluid flowing into the hollow hole of a hollow microneedle, a body fluid extraction channel is required. In addition to this problem, the sampled body fluid must be processed.

### SUMMARY OF THE DISCLOSURE

Therefore, the present disclosure has been made in view of the above problems, and it is an object of the present disclosure to provide a microneedle sensor capable of easily sensing specific substances in body fluid, such as blood glucose, in a minimally invasive manner using hollow microneedles and easily and continuously sensing changes in specific substances in body fluid, a method of manufacturing the microneedle sensor, and a continuous body fluid meter using the microneedle sensor.

It is another object of the present disclosure to provide a microneedle sensor capable of increasing the number of reaction electrodes per area by improving the installation structure of the reaction electrodes, a counter electrode, and a reference electrode and thus greatly improving the sensing range and accuracy of the microneedle sensor, a method of manufacturing the microneedle sensor, and a continuous body fluid meter using the microneedle sensor.

It is yet another object of the present disclosure to a microneedle sensor capable of increasing the reaction surface area of reaction electrodes by forming a fine roughness plating layer on the reaction electrodes and easily adjusting the sensing sensitivity of the reaction electrodes by changing the insertion length of the reaction electrodes inserted into the hollow holes of hollow microneedles, a method of manufacturing the microneedle sensor, and a continuous body fluid meter using the microneedle sensor.

In accordance with one aspect of the present disclosure, provided is a microneedle sensor including a sensor panel placed so that one surface thereof is in contact with skin of a body; a plurality of hollow microneedles that are formed on one surface of the sensor panel to protrude in form of micrometer-scale needles, wherein a hollow hole with one side exposed to outside is provided inside the hollow microneedle; reaction electrodes of a conductive material each inserted into the hollow holes of the hollow microneedles and configured to be inserted into an interior of the skin along with the hollow microneedles; a reference electrode of a conductive material applied to a first region formed on one surface of the sensor panel and applied to outer surfaces of hollow microneedles located on the first region; and a counter electrode of a conductive material applied to a second region formed on one surface of the sensor panel and applied to outer surfaces of hollow microneedles located on the second region.

Here, the first region and the second region may be provided at positions spaced apart from each other on one surface of the sensor panel. The hollow microneedles may be disposed in both the first and second regions.

In accordance with another aspect of the present disclosure, provided is a microneedle sensor including a sensor panel placed so that one surface thereof is in contact with skin of a body; a plurality of hollow microneedles that are formed on one surface of the sensor panel to protrude in form of micrometer-scale needles, wherein a hollow hole with one side exposed to outside is provided inside the hollow microneedle; reaction electrodes of a conductive material inserted into hollow holes of some of the hollow microneedles and configured to be inserted into an interior of the skin along with the hollow microneedles; a reference electrode of a conductive material inserted into a hollow hole of a remaining one of the hollow microneedles; and a counter electrode of a conductive material applied to one surface of the sensor panel and applied to outer surfaces of the hollow microneedles.

In accordance with still another aspect of the present disclosure, provided is a microneedle sensor including a sensor panel placed so that one surface thereof is in contact with skin of a body; a plurality of hollow microneedles that are formed on one surface of the sensor panel to protrude in form of micrometer-scale needles, wherein a hollow hole with one side exposed to outside is provided inside the hollow microneedle; reaction electrodes of a conductive material each inserted into the hollow holes of the hollow microneedles and configured to be inserted into an interior of the skin along with the hollow microneedles; and a reference electrode of a conductive material applied to one surface of the sensor panel and applied to outer surfaces of the hollow microneedles.

Preferably, on one side of the hollow microneedle, an opening may be formed by cutting the one side of the hollow microneedle. At this time, the one side of the hollow hole may be exposed to outside through the opening.

The hollow hole may be configured to extend long from an inside of the hollow microneedle to penetrate the sensor panel. The reaction electrodes may be formed as wires that penetrate the sensor panel and are inserted into the hollow holes.

Preferably, one end of the reaction electrode may be movably inserted into the hollow hole. Here, the reaction electrode may control sensing sensitivity of body fluid by adjusting a length of the hollow hole and changing a reaction area exposed through the opening.

Preferably, the opening may be formed in various sizes on one side of the hollow microneedle. Here, the reaction electrode may control sensing sensitivity of body fluid by changing a reaction area exposed through the opening depending on a size of the opening.

Preferably, the reaction electrode may include electrode wire of a conductive material with a fine roughness plating layer formed on a surface thereof; a sensor layer that is applied to one end of the electrode wire inserted into the hollow hole and includes a substance that reacts to specific sensing substances in body fluid; and a diffusion control layer applied to one end of the electrode wire to surround the sensor layer and configured to restrict passage of interfering substances within the body fluid that interferes with reaction with the specific sensing substances.

Here, the reaction electrode may further include a biocompatible membrane that is applied to surround the sensor layer and prevents biofouling due to an immune response when the hollow microneedles are inserted into skin; and an insulating layer that is applied to the other end of the electrode wire where the biocompatible membrane is not formed and insulates the electrode wire.

In addition, the sensor layer and the diffusion control layer may be applied by dip coating to one end of the electrode wire.

The sensor layer may include glucose oxidase (GOx) and an electron transfer mediator (osmium redox polymer) to measure blood glucose in the body fluid. The diffusion control layer may include Nafion.

In addition, on a surface of the electrode wire, a fine roughness plating layer with micropores may be formed depending on electrodeposition of Pt black (platinum powder).

Preferably, the reaction electrodes may be formed of any one of gold (Au), platinum (Pt), silver (Ag), carbon (C), copper (Cu), palladium (Pd), and stainless steel. The reference electrode may be formed of any one of standard hydrogen electrode (SHE), calomel (Hg/Hg₂Cl₂), and silver-silver chloride (Ag/AgCl). The counter electrode may be formed of any one of gold (Au), platinum (Pt), silver (Ag), carbon (C), copper (Cu), palladium (Pd), and stainless steel.

In accordance with still another aspect of the present disclosure, provided is a method of manufacturing a microneedle sensor, the method including manufacturing a sensor panel with a plurality of hollow microneedles formed on one surface thereof; forming a reference electrode by applying a conductive material to a first region formed on one surface of the sensor panel and outer surfaces of hollow microneedles located in the first region; forming a counter electrode by applying a conductive material to a second region formed on one surface of the sensor panel and outer surfaces of hollow microneedles located in the second region; manufacturing reaction electrodes of a conductive material in form of wires that are capable of being inserted into hollow holes of the hollow microneedles; and inserting the reaction electrodes into the hollow holes of the hollow microneedles to manufacture a microneedle sensor by combining the reaction electrodes and the hollow microneedles.

In accordance with still another aspect of the present disclosure, provided is a method of manufacturing a microneedle sensor, the method including manufacturing a sensor panel with a plurality of hollow microneedles formed on one surface thereof; forming a counter electrode by applying a conductive material to one surface of the sensor panel and outer surfaces of the hollow microneedles; manufacturing a reference electrode of a conductive material in form of a wire that is capable of being inserted into a hollow hole of the hollow microneedle; manufacturing reaction electrodes of a conductive material in form of wires that are capable of being inserted into hollow holes of the hollow microneedles; inserting the reaction electrodes into hollow holes of some of the hollow microneedles to manufacture a microneedle sensor by combining the reaction electrodes and the hollow microneedles; and inserting the reference electrode into a hollow hole of a remaining one of the hollow microneedles to manufacture a microneedle sensor by combining the reference electrode and the hollow microneedle.

In accordance with still another aspect of the present disclosure, provided is a method of manufacturing a microneedle sensor, the method including manufacturing a sensor panel with a plurality of hollow microneedles formed on one surface thereof; forming a reference electrode by applying a conductive material to one surface of the sensor panel and outer surfaces of the hollow microneedles; manufacturing reaction electrodes of a conductive material in form of wires that are capable of being inserted into hollow holes of the hollow microneedles; and inserting the reaction electrodes into the hollow holes of the hollow microneedles to manufacture a microneedle sensor by combining the reaction electrodes and the hollow microneedles.

Preferably, the manufacturing of the reaction electrodes may include preparing an electrode wire of a conductive material that is capable of being inserted into the hollow hole; forming a fine roughness plating layer on a surface of the electrode wire; applying a sensor layer that reacts with specific sensing substances of body fluid to the electrode wire formed on a surface of the fine roughness plating layer; and applying a diffusion control layer in a shape surrounding the sensor layer to the electrode wire to restrict passage of interfering substances in the body fluid that interfere with reaction with the specific sensing substances.

Here, the manufacturing of the reaction electrodes may further include applying a biocompatible membrane in a shape surrounding the diffusion control layer to the electrode wire to prevent biofouling due to an immune response when the hollow microneedles are inserted into skin; and applying an insulating layer to a surface of the electrode wire where the biocompatible membrane is not applied.

Preferably, the manufacturing of the reaction electrodes may further include testing sensing sensitivity of the microneedle sensor combined with the reaction electrodes and the hollow microneedles.

In accordance with yet another aspect of the present disclosure, provided is a continuous body fluid meter including the microneedle sensor.

Preferably, the continuous body fluid meter may further include a lower case having a sensor hole where the microneedle sensor is placed so that the hollow microneedles of the microneedle sensor are exposed to outside; a sensor board that is combined with the microneedle sensor to receive electrical signals from the microneedle sensor; a main board that is connected to the sensor board and continuously measures specific sensing substances in body fluid using electrical signals of the microneedle sensor; and an upper case coupled to the lower case to accommodate the main board, the sensor board, and the microneedle sensor therein.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a diagram schematically showing the configuration of a microneedle sensor according to this embodiment of the present disclosure;

FIG. 2 is a diagram schematically showing the operating state of the microneedle sensor shown in FIG. 1;

FIGS. 3 and 4 are perspective and top views showing the sensor panel and hollow microneedles shown in FIGS. 1 and 2;

FIG. 5 is a diagram showing insertion of a reaction electrode into the hollow microneedle shown in FIGS. 3 and 4;

FIG. 6 is a modified example of the hollow microneedle shown in FIG. 5, showing a reaction electrode inserted into the hollow microneedle;

FIG. 7 is a cross-sectional view showing the configuration of the reaction electrode shown in FIGS. 1 and 2;

FIG. 8 is a diagram showing the surface state of the reaction electrode shown in FIG. 7;

FIGS. 9 and 10 are graphs showing the performance of the reaction electrodes shown in FIG. 8, comparing reaction electrodes with a fine roughness plating layer and reaction electrodes without a fine roughness plating layer;

FIG. 11 is a flowchart schematically showing a method of manufacturing a microneedle sensor according to this embodiment of the present disclosure;

FIG. 12 is a diagram schematically showing the configuration of a microneedle sensor according to another embodiment of the present disclosure;

FIG. 13 a diagram schematically showing the configuration of a microneedle sensor according to another embodiment of the present disclosure;

FIGS. 14 to 16 are diagrams showing a continuous body fluid meter using a microneedle sensor according to this embodiment of the present disclosure; and

FIG. 17 is a graph comparing the performance of the continuous body fluid meter shown in FIGS. 14 to 16 and the performance of a conventional meter.

### DETAILED DESCRIPTION OF THE DISCLOSURE

Hereinafter, preferred embodiments of the present disclosure will be described in detail with reference to the accompanying drawings. However, the scope of the present disclosure is not limited by these embodiments. Like reference numerals in the drawings denote like elements.

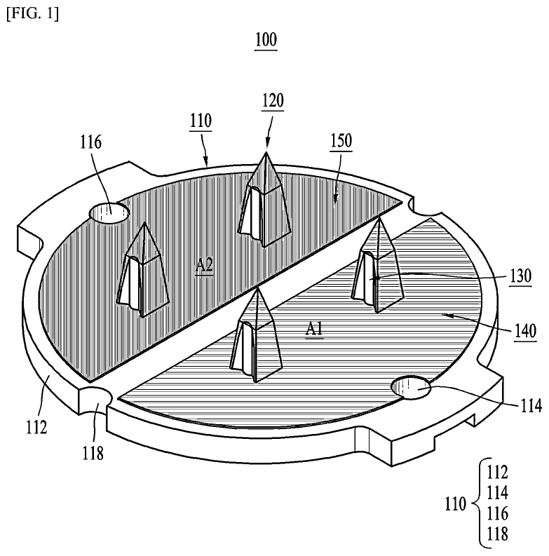

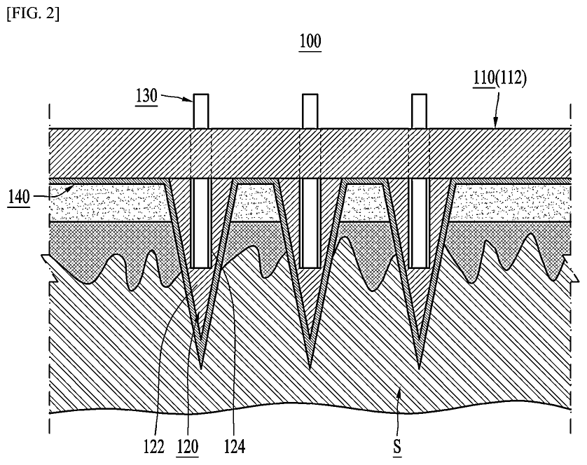

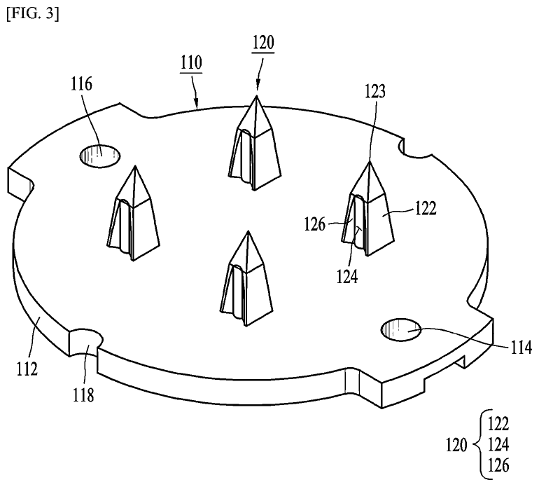

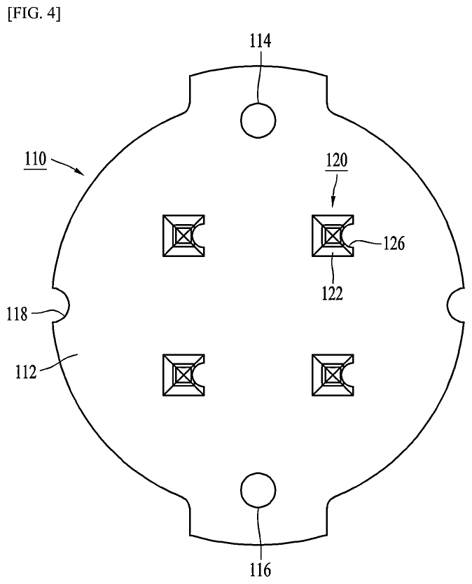

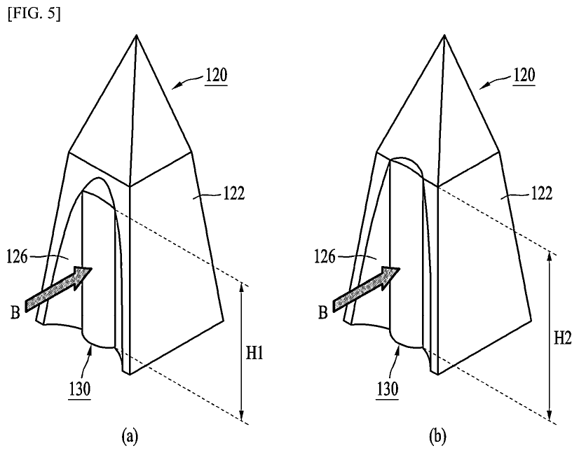

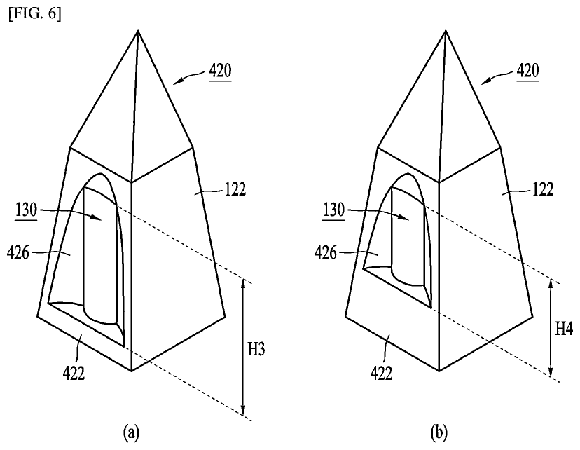

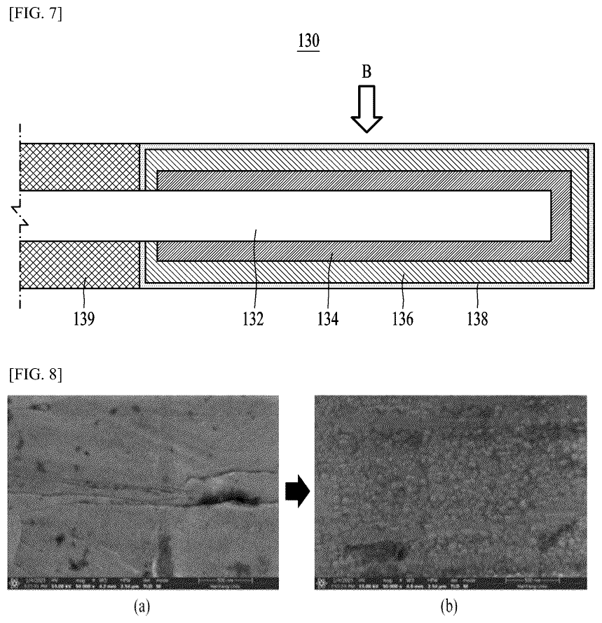

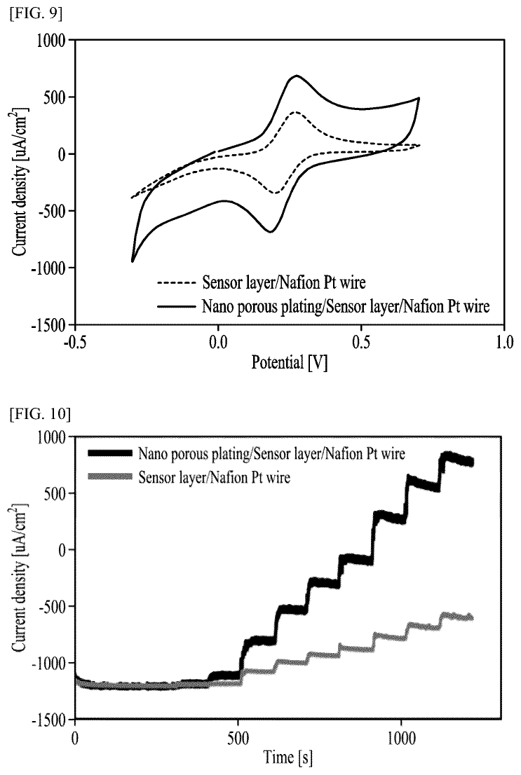

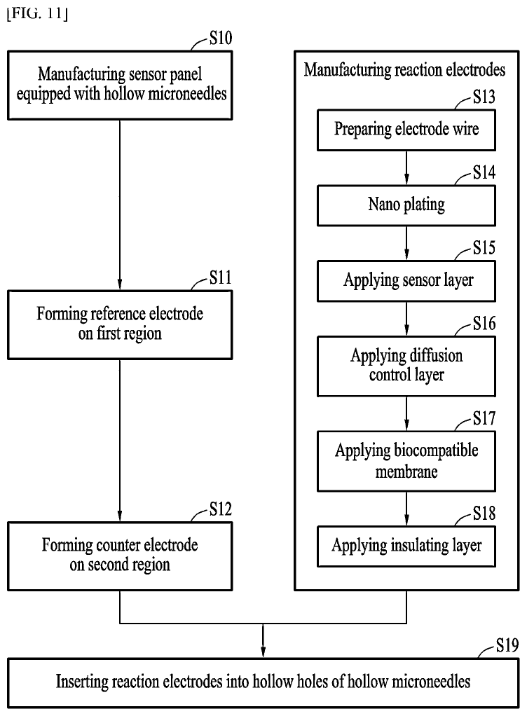

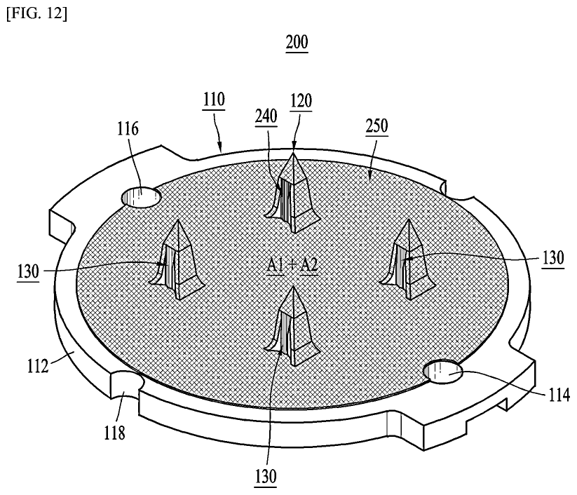

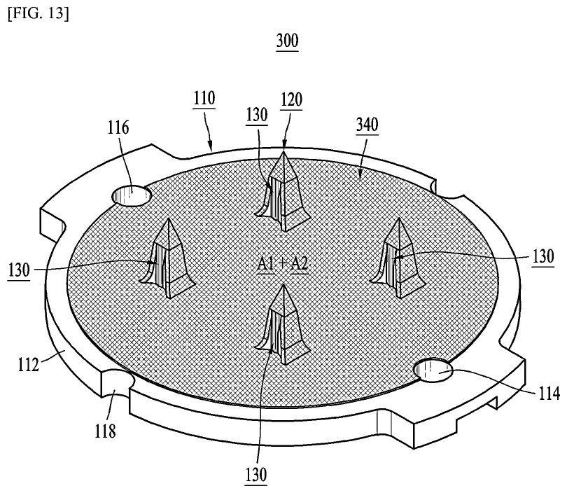

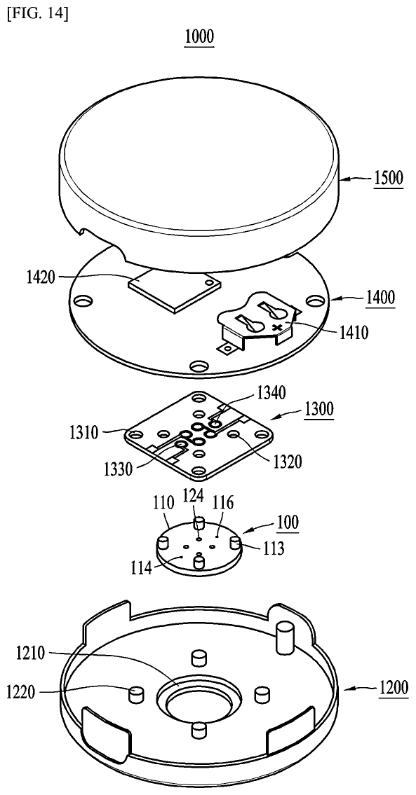

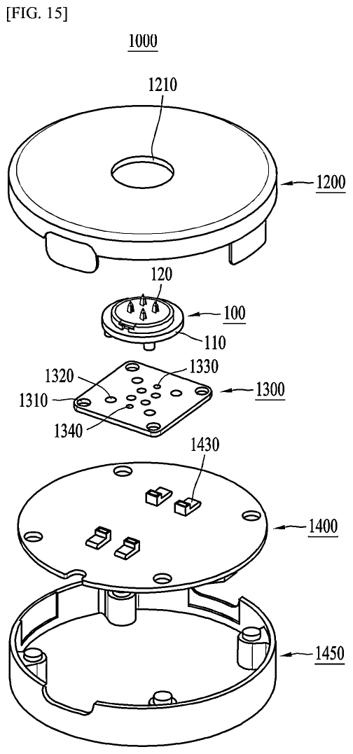

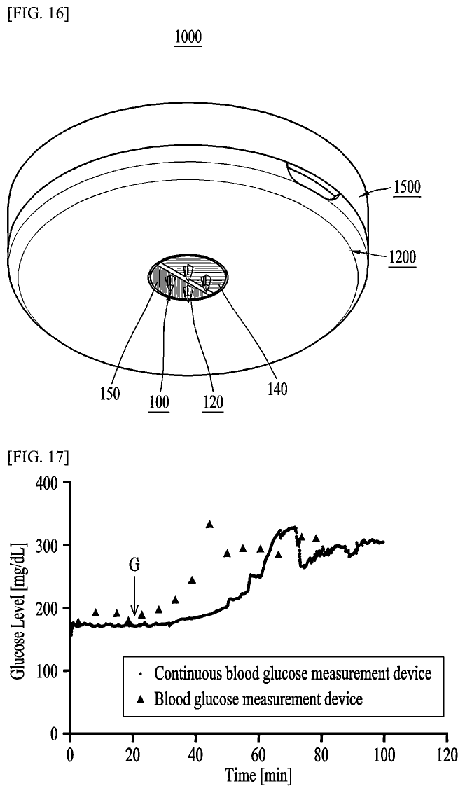

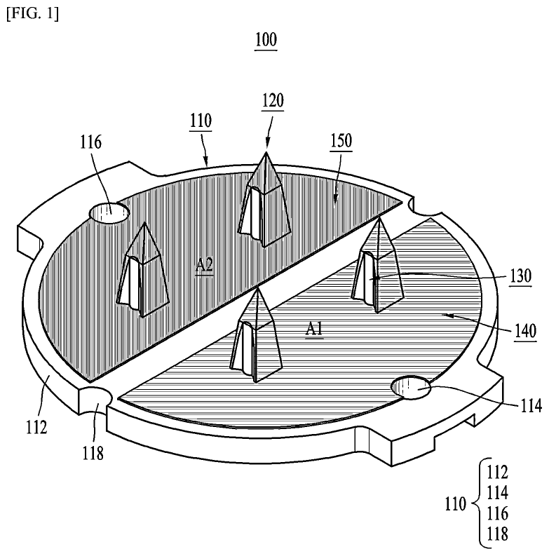

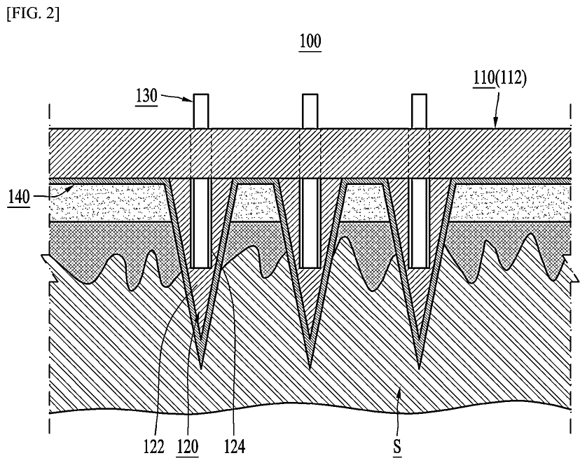

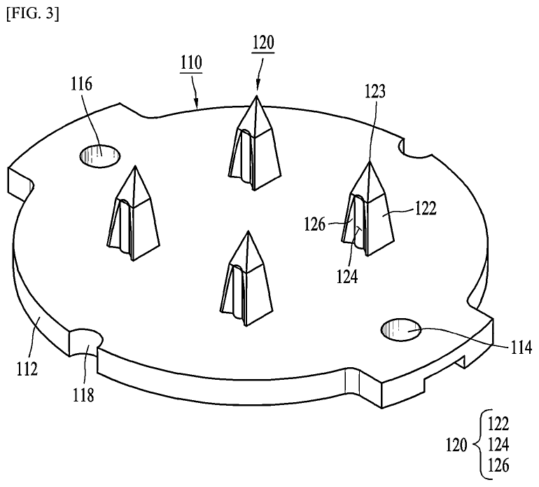

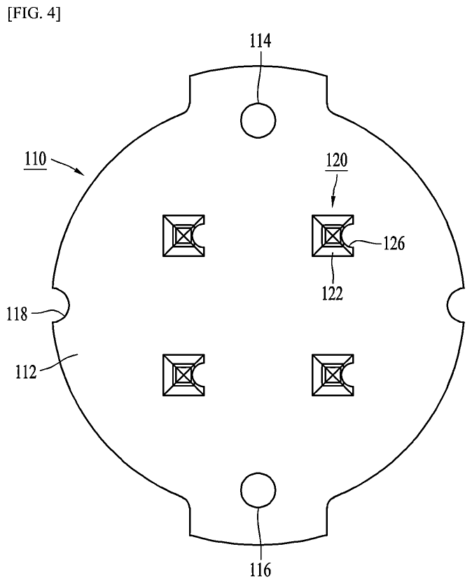

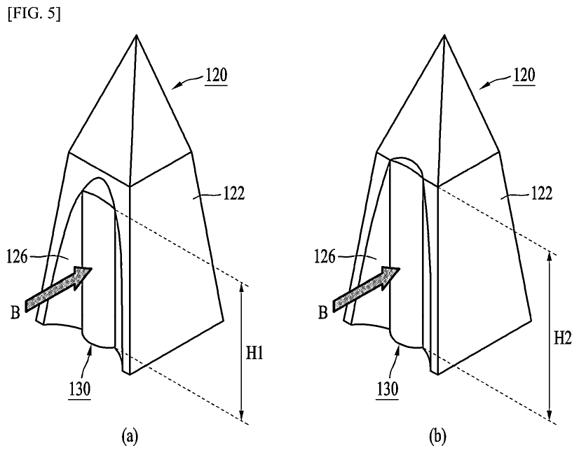

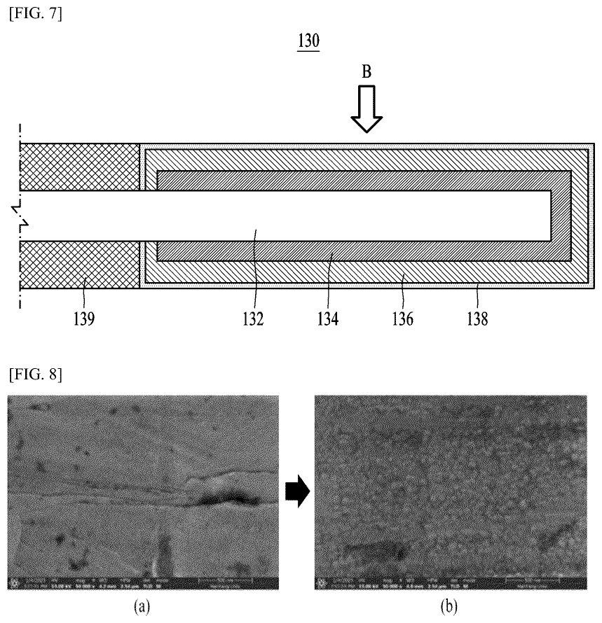

FIG. 1 is a diagram schematically showing the configuration of a microneedle sensor 100 according to this embodiment of the present disclosure, and FIG. 2 is a diagram schematically showing the operating state of the microneedle sensor 100 shown in FIG. 1. FIGS. 3 and 4 are perspective and top views showing a sensor panel 110 and hollow microneedles 120 shown in FIGS. 1 and 2, FIG. 5 is a diagram showing insertion of the reaction electrode into the hollow microneedle 120 shown in FIGS. 3 and 4, and FIG. 6 is a modified example of the hollow microneedle shown in FIG. 5, showing the reaction electrode inserted into the hollow microneedle. FIG. 7 is a cross-sectional view showing the configuration of the reaction electrode shown in FIGS. 1 and 2, and FIG. 8 is a diagram showing the surface state of the reaction electrode shown in FIG. 7.

Referring to FIGS. 1 and 2, the microneedle sensor 100 according to this embodiment of the present disclosure may include the sensor panel 110, the hollow microneedles 120, the reaction electrodes 130, a reference electrode 140, and a counter electrode 150.

The microneedle sensor 100 according to this embodiment is a biosensor attached to the skin(S) of the body and is capable of analyzing body fluid in a non-invasive manner using the hollow microneedles 120 and continuously measuring the specific sensing materials in body fluid such as interstitial fluid. Accordingly, when the microneedle sensor 100 according to this embodiment is applied to a body fluid meter (e.g., blood glucose meter) that measures the state of body fluid (e.g., blood glucose) by collecting body fluid (e.g., blood) using a conventional invasive manner, the state of body fluid may be continuously measured in a minimally invasive manner.

In addition, the microneedle sensor 100 according to this embodiment may use the hollow microneedles 120 to contact the reaction electrodes 130, the reference electrode 140, and the counter electrode 150 to body fluid, and then may analyze the electrical signals of the reaction electrodes 130, the reference electrode 140, and the counter electrode 150 to sense specific sensing substances in body fluid, such as glucose.

That is, when a plurality of hollow microneedles 120 are inserted into the skin(S) of the body, the reaction electrodes 130, the reference electrode 140, and the counter electrode 150 according to this embodiment may contact body fluid within the skin(S) and generate electrical signals depending on substances to be sensed in the body fluid, and may measure the concentration or presence of specific sensing substances in the body fluid by analyzing the electrical signals.

When the electrical signals of the reaction electrodes 130, the reference electrode 140, and the counter electrode 150 are used, a potentiostat mode or galvanostat mode may be used for specific sensing substances in body fluid. However, in this embodiment, a detailed description of a method of measuring body fluid using the reaction electrodes 130, the reference electrode 140, and the counter electrode 150 will be omitted.

Hereinafter, the microneedle sensor 100 according to this embodiment will be described as being formed in a panel shape that may be adhered to the skin(S) of the body, but the present disclosure is not limited thereto. The surfaces of the hollow microneedles 120 may be curved or stepped. In addition, the hollow microneedles 120 may be formed of a flexible material that may be deformed according to the shape of the skin(S) of the body.

Referring to FIGS. 1 to 4, the sensor panel 110 according to this embodiment may be formed in a panel shape that is in close contact with the skin(S) of the body. On one surface of the sensor panel 110, the hollow microneedles 120 may be provided in a protruding structure.

On one surface of the sensor panel 110, a first region A1 and a second region A2 may be provided at positions spaced apart from each other so as not to overlap each other. At this time, a plurality of hollow microneedles 120 may be disposed in the first and second regions A1 and A2 of the sensor panel 110, respectively. The shape, area, and arrangement pattern of the first and second regions A1 and A2 formed on one surface of the sensor panel 110 may vary depending on the design conditions and circumstances of the microneedle sensor 100.

Here, the second region A2 of the sensor panel 110 is a region where the counter electrode 150 is applied, and the first region A1 of the sensor panel 110 is a region where the reference electrode 140 is applied. Accordingly, the counter electrode 150 may be applied to the outer surfaces of the hollow microneedles 120 placed in the second region A2, and the reference electrode 140 may be applied to the outer surfaces of the hollow microneedles 120 placed in the first region A1.

For example, the sensor panel 110 may include a panel portion 112, a first terminal hole 114, a second terminal hole 116, and handle portions 118.

The panel portion 112 may be formed in a panel shape with a predetermined thickness. On one surface of the panel portion 112, the hollow microneedles 120 and the first and second regions A1 and A2 may be provided.

The first terminal hole 114 may be formed in the form of a through hole in the second region A2 of the sensor panel 110. For example, the first terminal hole 114 may be formed in the second region A2 of the sensor panel 110, and a first fastening terminal (not shown) made of a conductive material that serves as a terminal may be inserted into and fastened to the first terminal hole 114. Since the first fastening terminal is electrically connected to the counter electrode 150 applied to the surface of the second region A2, the first fastening terminal may serve as a passage for drawing electrical signals of the counter electrode 150 to the outside.

The second terminal hole 116 may be formed in the form of a through hole in the first region A1 of the sensor panel 110. For example, the second terminal hole 116 may be formed in the first region A1 of the sensor panel 110, and a second fastening terminal (not shown) made of a conductive material that serves as a terminal may be inserted into and fastened to the second terminal hole 116. Since the second fastening terminal is electrically connected to the reference electrode 140 applied to the surface of the first region A1, the second fastening terminal may serve as a passage for drawing electrical signals of the reference electrode 140 to the outside.

For reference, the first and second fastening terminals fastened to the first and second terminal holes 114 and 116 may serve as terminals for the reference electrode 140 and the counter electrode 150. Additionally, the first and second fastening terminals may serve to fix the panel portion 112 of the sensor panel 110 depending on the design conditions and circumstances of the microneedle sensor 100. Accordingly, the first and second fastening terminals may be provided as fastening members such as bolts or pins made of conductive materials.

The handle portions 118 may be provided on the panel portion 112 of the sensor panel 110 in a form that may be held by hand. For example, in this embodiment, it is explained that the handle portions 118 are provided in a groove shape at the edge of the panel portion 112 so that the handle portions 118 are used when assembling the sensor panel 110. However, the present disclosure is not limited thereto, and the shape and position of the handle portions 118 may be modified in various ways or the handle portions 118 may be omitted depending on the design conditions and circumstances of the microneedle sensor 100.

Referring to FIGS. 1 to 5, a plurality of hollow microneedles 120 according to this embodiment may protrude in the form of micrometer-scale needles on one surface of the sensor panel 110. The hollow microneedles 120 are the part that comes into contact with the body fluid within the skin(S) after being pierced into the skin(S) of the body. The hollow microneedles 120 may be placed in the first and second regions A1 and A2 of the sensor panel 110, respectively.

The hollow microneedles 120 according to this embodiment may be manufactured through a separate manufacturing process from the sensor panel 110 and then combined on one surface of the sensor panel 110. In addition, the hollow microneedles 120 may be manufactured as an integrated structure through the same manufacturing process as the sensor panel 110. Hereinafter, in this embodiment, the sensor panel 110 and the hollow microneedles 120 will be described as being manufactured as an integrated structure through a 3D printing process or injection molding process, but the present disclosure is not limited thereto and the sensor panel 110 and the hollow microneedles 120 may be manufactured through various processes.

In addition, the hollow microneedle 120 may include a needle body 122, a hollow hole 124, and an opening 126.

The needle body 122 may be formed to protrude from one surface of the sensor panel 110 in the shape of either a circular pyramid or a polygonal pyramid. The needle body 122 may be combined in an integrated structure on one surface of the sensor panel 110. In addition, a sharp needle tip 123 is formed at the upper end of the needle body 122, so that the needle body 122 may be smoothly inserted into the skin(S).

The hollow hole 124 may be provided inside the hollow microneedle 120 so that the reaction electrode 130 is inserted into the hollow hole 124. For this configuration, the hollow hole 124 may be formed to extend long inside the hollow microneedle 120 so as to penetrate the sensor panel 110.

The opening 126 may be formed on one side of the hollow microneedle 120 to expose the hollow hole 124 to the outside. That is, in this embodiment, one end of the reaction electrode 130 inserted into the hollow hole 124 may be exposed to the outside in a lateral direction (B) through the opening 126. When the hollow microneedle 120 is inserted into the skin(S), body fluid may flow in the lateral direction (B) through the opening 126 and react to one end of the reaction electrode 130. Accordingly, the reaction electrode 130 may increase or decrease the reaction area that reacts with specific sensing substances in the body fluid in proportion to the size of the opening 126.

Hereinafter, in this embodiment, it will be described that the opening 126 is formed on the side of the hollow microneedle 120 and is connected to communicate with the hollow hole 124. However, the present disclosure is not limited to thereto, and the opening 126 may be formed to communicate with the hollow hole 124 at the upper part of the needle body 122. However, as described in this embodiment, when the opening 126 is formed on the side of the hollow microneedle 120, the size of the opening 126 may further increase compared to the case where the opening 126 is formed on the upper part of the needle body 122. Additionally, a reaction area with the reaction electrode 130 may be increased by increasing body fluid flowing in through the opening 126. In addition, in this embodiment, since the upper part of the hollow hole 124 exists inside the upper part of the needle body 122, the reaction electrode 130 may be placed accurately and easily in a desired location through a simple method of inserting one end of the reaction electrode 130 until the one end catches the upper part of the hollow hole 124. In addition, by changing the position of the upper part of the hollow hole 124, the length at which the reaction electrode 130 is inserted into the hollow hole 124 may be changed.

For example, the opening 126 may be formed by cutting the side portion of the hollow microneedle 120. That is, the opening 126 may be connected to some of the sides of the hollow hole 124, and may be formed long along the longitudinal direction of the hollow hole 124. At this time, it is preferable that the reference electrode 140 and the counter electrode 150 are not applied to the opening 126.

As described above, when the opening 126 and the hollow hole 124 are formed in the needle body 122, the needle tip 123, which is sharply formed in the upper part of the needle body 122, remains, so the needle body 122 may be more easily inserted into the skin(S). In addition, when the reaction electrode 130 is inserted into the hollow hole 124, one end of the reaction electrode 130 may be caught on the ceiling of the upper part of the hollow hole 124, so insertion lengths H1 and H2 at which the reaction electrode 130 is maximally inserted may be stably limited.

Referring to FIG. 2 and FIGS. 5 to 8, the reaction electrodes 130 of this embodiment are electrodes that react with specific sensing substances in body fluid inside the skin(S), and may be inserted into the hollow holes 124 of the hollow microneedles 120, respectively. The reaction electrodes 130 are also commonly referred to as working electrodes. Accordingly, the reaction electrodes 130 may be inserted into the inside of the skin(S) together with the hollow microneedles 120 and may electrically react to specific sensing substances of body fluid flowing through the opening 126.

For this function, the reaction electrodes 130 may made of a conductive material and may be electrically connected to an external power source. For reference, in this embodiment, the reaction electrodes 130 are described as manufactured in the form of a wire of a conductive material that penetrates the sensor panel 110 and is inserted into the hollow hole 124.

For example, the reaction electrode 130 may include an electrode wire 132, a sensor layer 134, and a diffusion control layer 136.

The electrode wire 132 may be provided as a wire formed of a conductive material, and a fine roughness plating layer may be applied to the surface of the electrode wire 132. For example, the electrode wire 132 may be made of at least one of gold (Au), platinum (Pt), silver (Ag), carbon (C), copper (Cu), palladium (Pd), and stainless steel. In addition, a fine roughness plating layer with micropores may be formed on the surface of the electrode wire 132 by electrodeposition using Pt black (platinum powder). Hereinafter, in this embodiment, it will be described that the electrode wire 132 is formed of a material of Pt, and a fine roughness plating layer is formed on the surface of the electrode wire 132 by electrodeposition using Pt black (platinum powder).

FIG. 8A shows the surface of the electrode wire 132 without nanoporous plating, and FIG. 8B shows the surface of the electrode wire 132 with nanoporous plating. As shown in FIG. 8B, due to the fine roughness plating layer, surface roughness may be increased by a large number of micropores on the surface of the electrode wire 132. Due to the increase in surface roughness due to micropores, the reaction surface area of the electrode wire 132 that reacts with the body fluid may be greatly increased.

The sensor layer 134 is a coating layer containing a substance that react to specific sensing substances in body fluid. The sensor layer 134 may be applied to the surface of the electrode wire 132 inserted into the hollow hole 124, and may be applied to at least the surface of one end of the electrode wire 132. For example, the sensor layer 134 may include a substance (glucose oxidase (GOx) that reacts with glucose or glutamic acid dehydrogenase (GDH) and an electron transfer mediator (osmium redox polymer) to measure blood glucose in body fluid. Accordingly, the sensor layer 134 may generate an electrical signal in response to the glucose to be sensed, and may serve to transfer electrons generated during the reaction to the electrode wire 132.

The diffusion control layer 136 is a coating layer used to filter out only specific substances using a selectively transparent material, and may be applied to one end of the electrode wire 132 in a shape that surrounds the sensor layer 134. For example, the diffusion control layer 136 may include Nafion, which restricts the passage of interfering substances that interfere with the reaction of the sensor layer 134 and specific sensing substances in the body fluid. Accordingly, the diffusion control layer 136 may filter out interfering substances (e.g., acetaminophen, ascorbic acid, uric acid, etc.) in the body fluid and may transmit only necessary specific sensing substances (e.g., glucose) to the sensor layer 134.

For reference, one end of the electrode wire 132 may be coated with the sensor layer 134 and the diffusion control layer 136 by various methods such as drop coating, dip coating, spin coating, and electrospinning. Hereinafter, in this embodiment, it will be explained that the sensor layer 134 and the diffusion control layer 136 are applied to one end of the electrode wire 132 using a dip coating method in order to coat the surface of the electrode wire 132 with a constant thickness.

In addition, as shown in FIG. 7, the reaction electrodes 130 of this embodiment may further include a biocompatible membrane 138 and an insulating layer 139. In addition, the biocompatible membrane 138 and the insulating layer 139 of this embodiment may be omitted depending on the design conditions and circumstances of the microneedle sensor 100.

Here, the biocompatible membrane 138 may prevent biofouling from occurring on the outer surface of the hollow microneedles 120 due to an immune response after transplanting the hollow microneedles 120 to the skin(S) of a human body. For this function, the biocompatible membrane 138 may be applied to one end of the electrode wire 132 to surround the diffusion control layer 136. Accordingly, the biocompatible membrane 138 may stably ensure the performance of the reaction electrodes 130 in the human body by preventing degradation of sensing performance due to biofouling.

In addition, the insulating layer 139 may be formed of an insulating material to insulate the electrode wire 132 to prevent unnecessary interference and short circuit. The insulating layer 139 is preferably applied to insulate a portion of the reaction electrodes 130 inserted into the hollow hole 124 of the hollow microneedles 120 that is not exposed to the outside through the opening 126. For example, the insulating layer 139 may be applied to the surface of the other end of the electrode wire 132 on which the biocompatible membrane 138 is not formed.

In addition, as shown in FIGS. 2 and 5, the reaction electrode 130 may easily adjust sensing sensitivity for sensing specific sensing substances of body fluid by changing the insertion lengths H1 and H2 inserted into the hollow hole 124 of the hollow microneedle 120. That is, by changing the insertion lengths H1 and H2 of the reaction electrode 130, the surface area of the reaction electrode 130 exposed to the opening 126 of the hollow microneedle 120 may be increased or decreased, and accordingly, the reaction surface area where the reaction electrode 130 reacts with the body fluid may also be increased or decreased.

FIG. 2 shows a state in which the reaction electrodes 130 are inserted into the hollow holes 124 and then the reaction electrodes 130 are completely inserted up to the ceiling of the upper part of the hollow holes 124. The insertion lengths H1 and H2 of the reaction electrodes 130 are the lengths of inserting one end of the reaction electrodes 130 into the hollow holes 124 until the reaction electrodes 130 hang on the ceiling of the upper part of the hollow holes 124. The insertion lengths H1 and H2 may be changed by changing the length of the hollow holes 124.

That is, in this embodiment, the insertion lengths H1 and H2, at which the reaction electrode 130 is inserted up to the ceiling of the hollow hole 124, may be set according to the length of the hollow hole 124. As the insertion lengths H1 and H2 of the reaction electrode 130 increase, the reaction area of the reaction electrode 130 exposed to the opening 126 may also increase. Accordingly, in this embodiment, the insertion lengths H1 and H2 of the reaction electrode 130 may be set variously by changing the length of the hollow hole 124 formed inside the needle body 122. Accordingly, the sensing sensitivity of the body fluid may also be varied depending on the length of the hollow hole 124.

FIG. 5 shows the hollow microneedles 120 with different insertion lengths H1 and H2 of the reaction electrodes 130. That is, the hollow microneedles 120 shown in FIGS. 5A and 5B are the hollow microneedles 120 of different sizes that protrude at different heights. With this configuration, the size of the opening 126 may be increased or decreased in proportion to the size of the hollow microneedles 120.

Specifically, the hollow microneedle 120 shown in FIG. 5A may be formed to have a shorter height than the hollow microneedle 120 shown in FIG. 5B. With this configuration, the hollow hole 124 in the hollow microneedle 120 shown in FIG. 5A may be formed to have a shorter length than the hollow hole 124 in the hollow microneedle 120 shown in FIG. 5B, and the opening 126 of the hollow microneedle 120 shown in FIG. 5A is formed to have a shorter height than the opening 126 of the hollow microneedle 120 shown in FIG. 5B. Accordingly, the opening 126 shown in FIG. 5B may be formed to be larger than the opening 126 shown in FIG. 5A.

Accordingly, by designing and manufacturing the hollow microneedle 120 with the hollow hole 124 of various lengths, the size of the opening 126 may be varied. Accordingly, the reaction area of the reaction electrode 130 exposed to the outside through the opening 126 may also be set in various ways. That is, the reaction area of the reaction electrodes 130 exposed to the openings 126 may be easily changed by manufacturing the hollow microneedles 120 with the openings 126 of various sizes in advance and then replacing the hollow microneedles 120. Accordingly, the sensing sensitivity of body fluid may be easily adjusted.

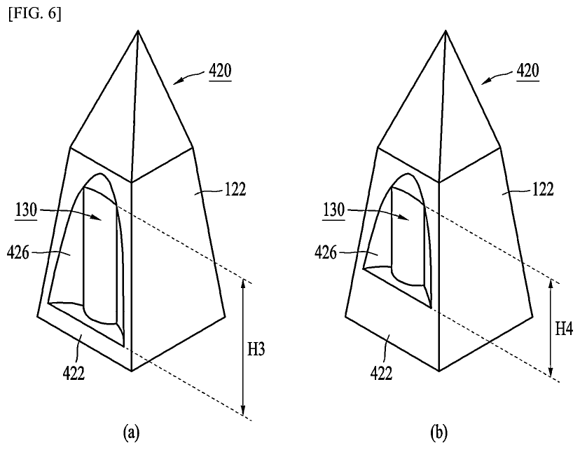

For reference, FIG. 6 shows a modified example of the hollow microneedles 120 according to this embodiment. That is, the hollow microneedles 420 of FIGS. 6A and 6B may be formed in the same size, but the opening 426 may be formed in different sizes on the side of the needle body 122. As shown in FIGS. 6A and 6B, the upper part of the opening 426 may be positioned equally at the upper part of the needle body 122 of the hollow microneedle 420, but the lower part of the opening 426 may be positioned at different heights from the lower part of the needle body 122.

Here, the opening 426 shown in FIG. 6A may be formed to be larger than the opening 426 shown in FIG. 6B. Accordingly, since the reaction area of the reaction electrode 130 is more exposed to the outside through the opening 426 shown in FIG. 6A, the hollow microneedle 420 shown in FIG. 6A may increase the sensing sensitivity of the body fluid to the reaction electrode 130 more than the hollow microneedle 420 shown in FIG. 6B.

For example, the hollow microneedles 420 of this modification may be easily manufactured by manufacturing the hollow microneedles 120 shown in FIG. 5 in advance and then shielding the lower part of the opening 426 of the hollow microneedles 120 with a shield wall 422 of an insulating material. Accordingly, in the hollow microneedles 420 of this modification, the opening 426 of various heights H3 and H4 may be formed by adjusting the area that shields the lower part of the opening 126 shown in FIG. 5 with the shield wall 422 of an insulating member. The size of the opening 426 may be easily set by changing the heights H3 and H4 of the opening 426. The sensing sensitivity of the body fluid to the reaction electrode 130 may be easily adjusted by changing the reaction area of the reaction electrode 130 exposed to the opening 426 according to the size of the opening 426.

Referring to FIGS. 1 and 2, the counter electrode 150 of this embodiment is an electrode through which current flows between the reaction electrodes 130. The counter electrode 150 is also commonly referred to as a counter electrode. In addition, the conventional counter electrode is generally placed in the hollow hole 124 of the hollow microneedle 120 like the reaction electrode 130, but in this embodiment, the counter electrode 150 may be applied in a thin layer to the second region A2 of the sensor panel 110.

Specifically, the counter electrode 150 may be applied to the second region A2 of the sensor panel 110, and may also be applied to the outer surface of the hollow microneedles 120 located in the second region A2. Accordingly, when the microneedle sensor 100 is in close contact with the skin(S) of the body, the counter electrode 150 is also inserted into the inside of the skin(S) together with the hollow microneedles 120 and may come into contact with body fluid.

The counter electrode 150 may be formed of the same conductive material as the reaction electrodes 130. For example, the counter electrode 150 may be provided as a plating layer formed of at least one of gold (Au), platinum (Pt), silver (Ag), carbon (C), copper (Cu), palladium (Pd), and stainless steel.

Referring to FIGS. 1 and 2, the reference electrode 140 of this embodiment is an electrode that represents a potential of a certain size as a standard for analyzing the measured values of the reaction electrodes 130 and the counter electrode 150. The reference electrode 140 is also commonly referred to as a reference electrode. In addition, the conventional reference electrode is generally placed in the hollow hole 124 of the hollow microneedles 120 like the reaction electrodes 130. However, in this embodiment, the reference electrode 140 may be applied in a thin layer to the first region A1 of the sensor panel 110 to correspond to the counter electrode 150.

Specifically, the reference electrode 140 may be applied to the first region A1 of the sensor panel 110, and may also be applied to the outer surface of the hollow microneedles 120 located in the first region A1. Accordingly, when the microneedle sensor 100 is in close contact with the skin(S) of the body, the reference electrode 140 may be also inserted into the skin(S) together with the hollow microneedles 120 and may come into contact with body fluid.

The reference electrode 140 may be formed of a conductive material. For example, the reference electrode 140 may be formed of any one of standard hydrogen electrode (SHE), calomel (Hg/Hg₂Cl₂), and silver-silver chloride (Ag/AgCl). Preferably, in this embodiment, the reference electrode 140 may be provided as a plating layer made of Ag/AgCl.

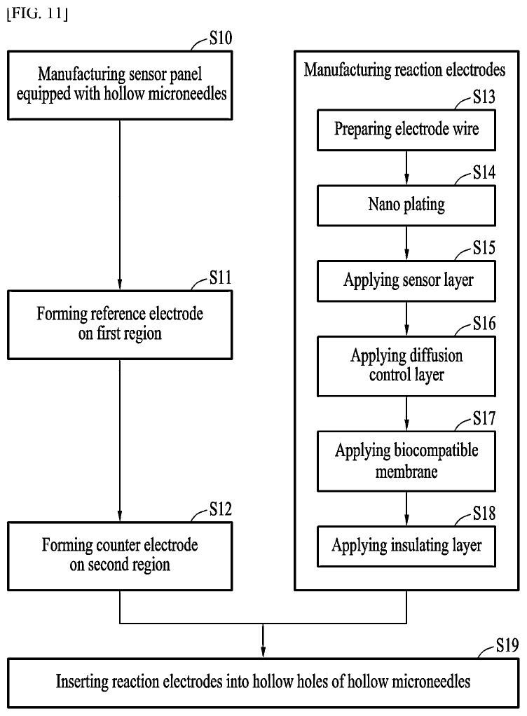

FIG. 11 is a diagram schematically showing a method of manufacturing the microneedle sensor 100 according to this embodiment of the present disclosure.

The method of manufacturing the microneedle sensor 100 according to this embodiment of the present disclosure will be described as follows.

Referring to FIG. 11, the method of manufacturing the microneedle sensor 100 according to this embodiment of the present disclosure may include a step of manufacturing the sensor panel 110 with the hollow microneedles 120 formed on one side thereof (see S10), a step of forming the reference electrode 140 by applying a conductive material to the first region A1 formed on one surface of the sensor panel 110 and the outer surfaces of the hollow microneedles 120 located at the first region A1 (see S11), a step of forming the counter electrode 150 by applying a conductive material to the second region A2 formed on one surface of the sensor panel 110 and the outer surfaces of the hollow microneedles 120 located at the second region A2 (see S12), a step of manufacturing the reaction electrodes 130 of a conductive material in the form of wires that are capable of being inserted into the hollow holes 124 of the hollow microneedles 120 (see S13 to S16), a step of inserting the reaction electrodes 130 into the hollow holes 124 of the hollow microneedles 120 to manufacture the microneedle sensor 100 by combining the reaction electrodes 130 and the hollow microneedles 120 (see S17), and a step of testing the sensing sensitivity of the reaction electrodes 130 (see S18).

In the step of manufacturing the sensor panel 110 equipped with the hollow microneedles 120 (see S10), the hollow microneedles 120 and the sensor panel 110 are manufactured in an integrated structure. That is, the hollow microneedles 120 and the sensor panel 110 are molded using the same material through a 3D printing process or injection molding process.

In the step of forming the reference electrode 140 on the first region A1 (see S11), Ag/AgCl material is applied at a preset thin thickness to the first region A1 provided on one surface of the sensor panel 110 to form the reference electrode 140. Accordingly, the reference electrode 140 may be provided as a coating layer applied to the surface of the first region A1 of the sensor panel 110, and may also be applied to the outer surfaces of the hollow microneedles 120 located in the first region A1.

In the step of forming the counter electrode 150 on the second region A2 (see S12), Pt material is applied at a preset thin thickness to the second region A2 provided on one surface of the sensor panel 110 to form the counter electrode 150. Accordingly, the counter electrode 150 is provided as a coating layer applied to the surface of the second region A2 of the sensor panel 110, and is preferably also applied to the outer surfaces of the hollow microneedles 120 located in the second region A2.

In the step of manufacturing the reaction electrodes 130 (see S13 to S16), the reaction electrodes 130 are manufactured in the form of a wire made of Pt that is capable of being inserted into the hollow hole 124 of the hollow microneedles 120. After forming a fine roughness plating layer, the sensor layer 134 and the diffusion control layer 136 are coated.

For example, the step of manufacturing the reaction electrodes may include a step of preparing, using Pt material, the electrode wire 132 that is capable of being inserted into the hollow hole 124 of the hollow microneedle 120 (see S13), a step of forming a fine roughness plating layer on the surface of the electrode wire 132 (see S14), a step of applying the sensor layer 134, which reacts with specific sensing substances of body fluid, to the electrode wire 132 formed on the surface of the fine roughness plating layer (see S15), and a step of applying the diffusion control layer 136 in a shape surrounding the sensor layer 134 to the electrode wire 132 to restrict the passage of interfering substances in the body fluid that interfere with the reaction with the specific sensing substances (see S16).

Here, the step (S14) of forming a fine roughness plating layer may be performed in the order of 1) to 5) described below.

1) Chloroplatinic acid hydrate, 0.3 M sodium chloride (NaCl) solution, and Triton X-100 are mixed at 60° C.

2) After boiling the solution prepared in 1) at 40° C., an Ag/AgCl electrode and the electrode wire 132 are immersed in the solution. Then, the Ag/AgCl electrode is connected to a reference electrode of a potentiostat, and the electrode wire 132 is connected to a reaction electrode of a potentiostat.

3) After forming a fine roughness plating layer by applying −150 μA for 50 minutes using a constant current mode of a potentiostat, washing is performed a total of 3 times for 1 hour each using distilled water.

4) In a 1 M sulfuric acid solution, the electrode wire 132 on which the fine roughness plating layer prepared in 3) is formed is connected to the reaction electrode of a potentiostat, and an Hg/Hg₂SO₄ electrode is connected to the reference electrode of a potentiostat. Then, washing is performed 20 times in the range of −0.72 to 0.68 V using cyclic voltammetry of a potentiostat.

5) The electrode wire 132 manufactured in step 4) is washed with distilled water and dried, and then the electrode wire 132 made of Pt with a fine roughness plating layer formed on the surface is manufactured.

In addition, in the step (S15) of applying the sensor layer 134 and the step (S16) of applying the diffusion control layer 136, using the dip coating method, the surface of the electrode wire 132 is coated with the sensor layer 134 and the diffusion control layer 136 to a uniform thickness. At this time, the diffusion control layer 136 is coated on the electrode wire 132 to completely surround the sensor layer 134.

The step (S15) of applying the sensor layer 134 and the step (S16) of applying the diffusion control layer 136 may be performed in the order of 11) to 19) described below. That is, the dip coating solution of the sensor layer 134 is prepared through the processes of 11) to 14), the sensor layer 134 is coated through the processes of 15) to 17), and the diffusion control layer 136 is coated through the processes of 18) to 20).

11) 7 mg of polyvinylimidazole-osmium (PVI-Os) is dissolved in 100 μl of ethanol.

12) 75 mg of PEGDGE is dissolved in 500 μl of pure DI water.

13) 6 mg of GOx is dissolved in 100 μl of pure water.

14) 53 μl of the aqueous PVI-Os solution prepared in 11), 6 μl of the aqueous PEGDGE solution prepared in 12), and 40 μl of the aqueous GOx solution prepared in 13) are mixed to prepare a sensor layer coating solution.

15) The sensor layer 134 is applied by dip-coating the electrode wire 132 made of Pt into the sensor layer coating solution prepared in 14).

16) The manufacturing of the reaction electrodes 130 is completed by dip-coating the electrode wire 132 coated with the sensor layer 134 in an Nafion stock solution and applying the diffusion control layer 136. In addition, depending on the design conditions and circumstances of the microneedle sensor 100, the biocompatible membrane 138 coated on the outer surface of the diffusion control layer 136 and the insulating layer 139 coated on areas that do not need to react with the body fluid of the reaction electrodes 130 may be added.

In the step (S17) of inserting the reaction electrodes 130 into the hollow holes 124 of the hollow microneedles 120, the reaction electrodes 130 are each inserted into the hollow holes 124 of the hollow microneedles 120, with the insertion lengths H1 and H2 corresponding to the length of the hollow holes 124. Manufacture of the microneedle sensor 100 is completed by combining the reaction electrodes 130 and the hollow microneedles 120.

In addition, the method of manufacturing the microneedle sensor 100 according to this embodiment may further include a step of testing the sensing sensitivity of the microneedle sensor 100 in which the reaction electrodes 130 and the hollow microneedles 120 are combined. For example, using test equipment, preset power is provided to the reaction electrodes 130, the reference electrode 140, and the counter electrode 150 of the microneedle sensor 100, and measurements derived from the reaction electrodes 130, the reference electrode 140, and the counter electrode 150 are transmitted to the test equipment and checked for errors.

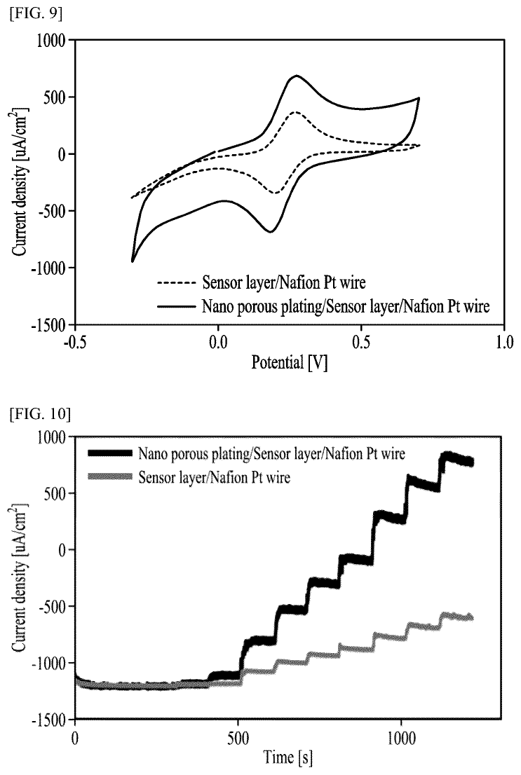

FIGS. 9 and 10 are graphs showing the performance of the reaction electrodes 130 shown in FIG. 8, and show the results of comparing the reaction electrodes 130 on which a fine roughness plating layer is formed and the reaction electrodes on which a fine roughness plating layer is not formed.

Referring to FIG. 9, the reaction electrodes 130 on which a fine roughness plating layer is formed exhibits a higher redox peak than the reaction electrodes on which a fine roughness plating layer is not formed. That is, the reaction surface area of the reaction electrodes 130 with a fine roughness plating layer formed thereon, which reacts with the body fluid, is greater than that of the reaction electrodes without nanoporous plating, indicating that the sensing sensitivity of the reaction electrodes 130 is improved.

Referring to FIG. 10, when the reaction electrodes 130 with a fine roughness plating layer formed thereon is compared with the reaction electrodes without a fine roughness plating layer, the level of response to glucose increases by more than 3 times. That is, since the reaction electrodes 130 with a fine roughness plating layer formed thereon reacts more easily with glucose contained in the body fluid than the reaction electrodes without a fine roughness plating layer, the reaction electrodes 130 with a fine roughness plating layer formed thereon is very useful in measuring blood glucose. Based on these results, it can be seen that the sensing sensitivity of the reaction electrodes 130 to glucose is also improved because the reaction surface area of the reaction electrodes 130 increases.

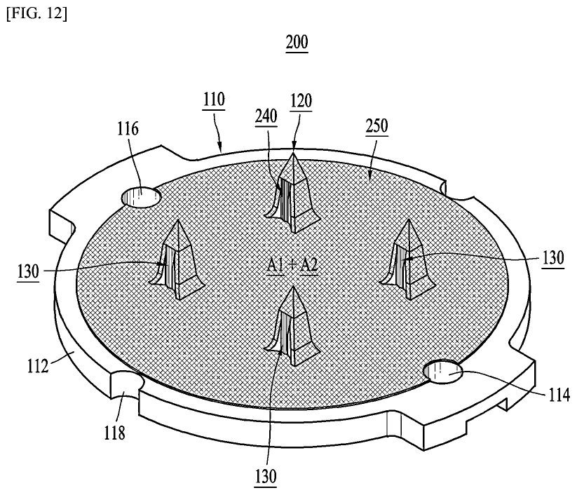

FIG. 12 is a diagram schematically showing the configuration of a microneedle sensor 200 according to another embodiment of the present disclosure.

In FIG. 12, reference numbers that are the same or similar to those shown in FIGS. 1 to 11 indicate the same members, and detailed descriptions thereof will be omitted. Hereinafter, the description will focus on differences from the microneedle sensor 100 shown in FIGS. 1 to 11.

Referring to FIG. 12, the difference between the microneedle sensor 200 according to another embodiment of the present disclosure and the microneedle sensor 100 shown in FIGS. 1 to 11 is that the arrangement structure of the reaction electrodes 130, a reference electrode 240, and a counter electrode 250 are different.

The reaction electrodes 130 of this embodiment are prepared in the same way as the reaction electrodes 130 shown in FIGS. 1 to 11, but the difference is that the reaction electrodes 130 are inserted into the hollow holes 124 of some of the hollow microneedles 120. That is, in this embodiment, the reaction electrodes 130 may be inserted into the hollow holes 124 of three of the four hollow microneedles 120.

The reference electrode 240 of this embodiment may be inserted into the hollow hole 124 of a remaining one of the hollow microneedles 120. Accordingly, in this embodiment, the reference electrode 240 may be manufactured in the same manner as the reaction electrodes 130 in the form of a wire of conductive material. The reference electrode 240 may be inserted into the hollow hole 124 of one hollow microneedle 120 where the reaction electrodes 130 are not inserted among the four hollow microneedles 120.

The counter electrode 250 of this embodiment has differences in the application area for the sensor panel 110 and the hollow microneedles 120 compared to the counter electrode 150 shown in FIGS. 1 to 11, and other structures and materials may be prepared in the same manner. That is, in this embodiment, the counter electrode 250 may be formed in a structure in which the counter electrode 250 is applied to the entire one surface of the sensor panel 110 and the outer surfaces of the hollow microneedles 120.

When comparing with the microneedle sensor 100 shown in FIGS. 1 to 11, in the microneedle sensor 200 shown in FIG. 12, the sensitivity of the reaction electrodes 130 may be relatively low. However, since the microneedle sensor 200 shown in FIG. 12 has a structure in which the reference electrode 240 is inserted into the skin(S) together with the hollow microneedles 120, the sensitivity of the reference electrode 240 may be stably secured. In addition, since the counter electrode 250 is applied both to the entire surface of the sensor panel 110, which combines the first and second regions A1 and A2 shown in FIGS. 1 to 11, and the outer surfaces of the hollow microneedles 120, the sensitivity of the counter electrode 250 may be improved.

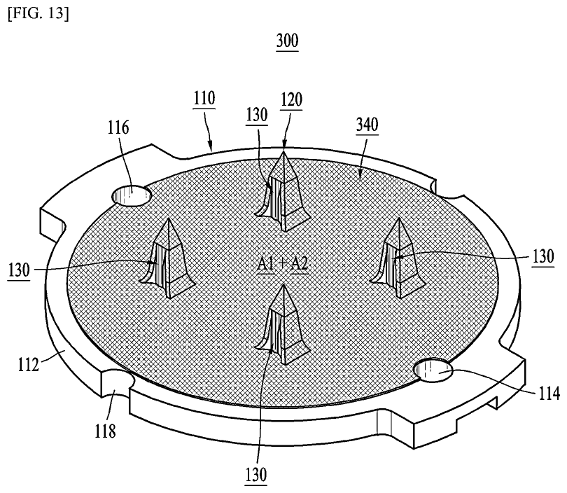

FIG. 13 is a diagram schematically showing the configuration of a microneedle sensor 300 according to another embodiment of the present disclosure.

In FIG. 13, reference numbers that are the same or similar to those shown in FIGS. 1 to 11 indicate the same members, and detailed descriptions thereof will be omitted. Hereinafter, the description will focus on differences from the microneedle sensor 100 shown in FIGS. 1 to 11.

Referring to FIG. 13, the difference between the microneedle sensor 300 according to another embodiment of the present disclosure and the microneedle sensor 100 shown in FIGS. 1 to 11 is that the counter electrode is omitted and the arrangement of a reference electrode 340 is different.

The microneedle sensor 300 of this embodiment may be provided in a structure in which the counter electrode 150 shown in FIGS. 1 to 11 is not used. That is, the microneedle sensor 100 shown in FIGS. 1 to 11 is composed of a three-electrode type using the reaction electrodes 130, the reference electrode 140, and the counter electrode 150, but the microneedle sensor 300 of this embodiment is composed of a two-electrode type using the reaction electrodes 130 and the reference electrode 140.

Here, since the reaction electrodes 130 of this embodiment are provided in the same manner as the reaction electrodes 130 shown in FIGS. 1 to 11, description thereof will be omitted.

In addition, the reference electrode 340 of this embodiment has differences in application sites for the sensor panel 110 and the hollow microneedles 120 compared to the reference electrode 140 shown in FIGS. 1 to 11, and other structures and materials may be prepared in the same manner. That is, in this embodiment, the reference electrode 340 may be applied to the entire surface of one surface of the sensor panel 110 and the outer surfaces of the hollow microneedles 120.

Compared to the microneedle sensor 100 shown in FIGS. 1 to 11, the microneedle sensor 300 shown in FIG. 13 has the disadvantage of not being able to use the counter electrode 150. However, since the reference electrode 340 is applied to the entire one surface of the sensor panel 110 including the first and second regions A1 and A2 shown in FIGS. 1 to 11 and the outer surfaces of the hollow microneedles 120, the sensitivity of the reference electrode 340 may be improved.

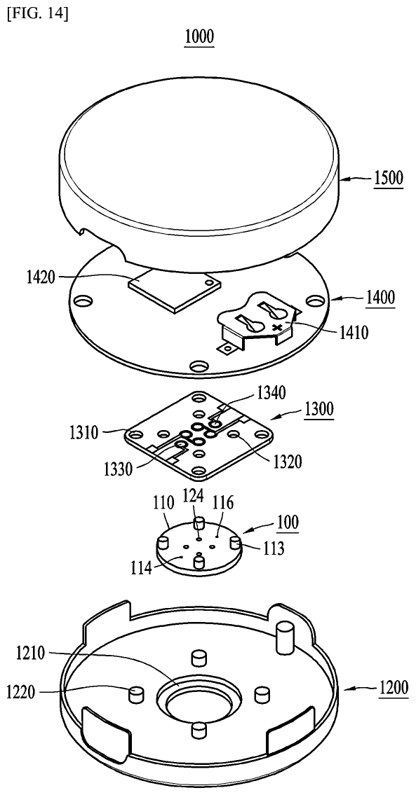

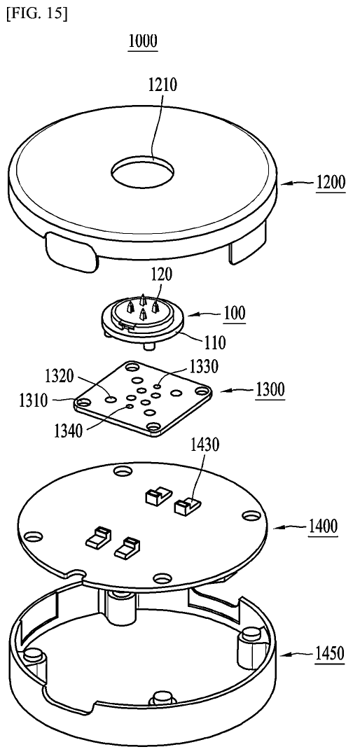

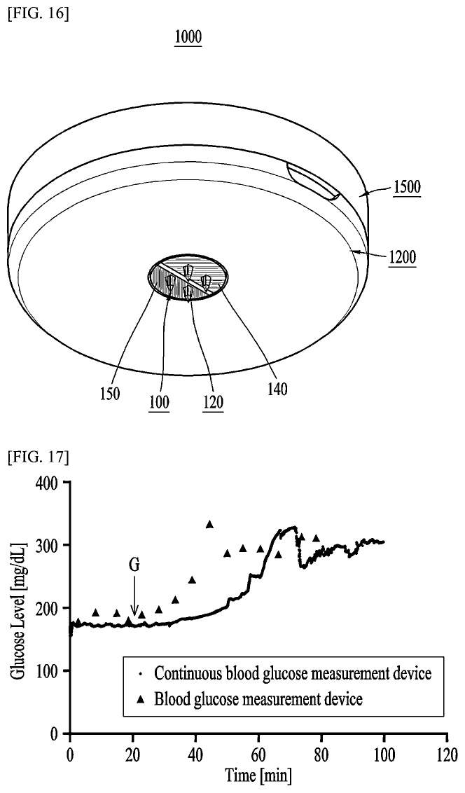

FIGS. 14 to 16 are diagrams showing a continuous body fluid meter 1000 using the microneedle sensor 100 according to this embodiment of the present disclosure, and FIG. 17 is a graph comparing the performance of the continuous body fluid meter 1000 shown in FIGS. 14 to 16 and the performance of the existing measuring device.

Referring to FIGS. 14 to 16, the continuous body fluid meter 1000 according to an embodiment of the present disclosure may continuously measure specific sensing substances contained in body fluid within the skin(S) using the microneedle sensor 100 shown in FIGS. 1 to 11, and may analyze the state of the body fluid according to the measured values of the specific sensing substances. Hereinafter, in this example, the continuous body fluid meter 1000 will be described as a continuous blood glucose meter that measures glucose in body fluid and analyzes changes in blood glucose. In addition, in this embodiment, for convenience of explanation, the vertical direction is defined based on the microneedle sensor 100 in FIG. 14. For example, as shown in FIG. 14, an upper case 1500 is located at the top, and a lower case 1200 is located at the bottom.

As described above, the continuous body fluid meter 1000 may be structured to measure a specific sensing substance (e.g., glucose) of body fluid using the microneedle sensor 100. For reference, in FIGS. 14 to 16, reference numbers that are the same or similar to those shown in FIGS. 1 to 11 indicate the same members, and detailed descriptions thereof will be omitted.

For example, the continuous body fluid meter 1000 of this embodiment may include the microneedle sensor 100, the lower case 1200, a sensor board 1300, a main board 1400, and the upper case 1500.

As shown in FIGS. 14 to 16, the microneedle sensor 100 according to this embodiment may be configured identically to the microneedle sensor 100 shown in FIGS. 1 to 11, and detailed description thereof will be omitted. Hereinafter, in this embodiment, since the continuous body fluid meter 1000 is a continuous blood glucose meter for continuous measurement of blood glucose, the microneedle sensor 100 may also be provided to measure the concentration of glucose.

On the upper surface of the sensor panel 110 of the microneedle sensor 100,

A plurality of needle sensor guide portions 113 may be formed to be respectively connected to a plurality of needle sensor connection grooves 1320, which will be described later, formed on the sensor board 1300. For example, the needle sensor guide portions 113 may protrude as cylindrical protrusions and be respectively coupled to the needle sensor connection grooves 1320.

As shown in FIGS. 14 to 16, the lower case 1200 of this embodiment may be formed to accommodate the microneedle sensor 100. The lower case 1200 may be tightly attached to the skin(S) of the body when using the continuous body fluid meter 1000.

A sensor hole 1210 for placing the microneedle sensor 100 may be formed in the lower case 1200. That is, the microneedle sensor 100 may be placed inside the lower case 1200. At this time, the microneedle sensor 100 may be seated and fixed in the sensor hole 1210 of the lower case 1200 so that the hollow microneedles 120 are exposed to the outside through the sensor hole 1210.

The bottom of the lower case 1200 may be coated with an adhesive layer (not shown) to adhere to the skin(S), and a plurality of sensor board guide portions 1220 may be provided on the inner bottom of the lower case 1200 to guide the installation of the sensor board 1300. For example, the sensor board guide portions 1220 may protrude as cylindrical protrusions and may be respectively coupled to sensor board installation holes 1310 formed on the sensor board 1300, which will be described later.

As shown in FIGS. 14 to 16, the sensor board 1300 of this embodiment may be coupled to the upper surface of the microneedle sensor 100 and may be electrically connected to the reaction electrodes 130, the reference electrode 140, and the counter electrode 150. That is, the sensor board 1300 may apply electricity to the reaction electrodes 130, the reference electrode 140, and the counter electrode 150, and then may receive electrical signals of the reaction electrodes 130, the reference electrode 140, and the counter electrode 150. The sensor board 1300 may be manufactured in a pre-coupled state with the microneedle sensor 100 and may be treated as one part together with the microneedle sensor 100.

At the edge of the sensor board 1300, the sensor board installation holes 1310 may be formed at positions corresponding to the sensor board guide portions 1220 of the lower case 1200, and the needle sensor connection grooves 1320 may be formed inside the sensor board installation holes 1310 to connect the needle sensor guide portions 113 of the microneedle sensor 100.

At this time, the sensor board 1300 and the microneedle sensor 100 may be stably coupled to each other by combining the needle sensor connection grooves 1320 and the needle sensor guide portions 113, and the sensor board 1300 may be stably fixed inside the lower case 1200 by combining the sensor board installation holes 1310 and the sensor board guide portions 1220.

In addition, in the center of the sensor board 1300, first electrode connection circuit holes 1330 may be formed to electrically connect the reaction electrodes 130 of the microneedle sensor 100, and second electrode connection circuit holes 1340 may be formed to electrically connect the reference electrode 140 and the counter electrode 150 of the microneedle sensor 100. At this time, the first electrode connection circuit holes 1330 may be formed at positions corresponding to the hollow holes 124 into which the reaction electrodes 130 of the microneedle sensor 100 are inserted. In addition, the second electrode connection circuit holes 1340 may be formed at positions corresponding to the first terminal hole 114 and the second terminal hole 116, which are electrically connected to the reference electrode 140 and the counter electrode 150 of the microneedle sensor 100.

As shown in FIGS. 14 to 16, the main board 1400 of this embodiment may continuously measure blood glucose using electrical signals detected by the reaction electrodes 130, the reference electrode 140, and the counter electrode 150. For this function, the main board 1400 and the sensor board 1300 may be individually manufactured and then electrically connected to each other when the continuous body fluid meter 1000 is assembled.

For example, in this embodiment, a plurality of terminal connection portions 1430 may be formed on the bottom of the main board 1400, and the terminal connection portions 1430 may be formed to be electrically connected to each other when the main board 1400 and the sensor board 1300 are connected. Circuit terminals electrically connected to the first electrode connection circuit holes 1330 and the second electrode connection circuit holes 1340 may be formed on the sensor board 1300, respectively. The terminal connection portions 1430 may be individually contacted to each circuit terminal portion. The terminal connection portions 1430 may be provided to be elastically pressable for stable connection.

In addition, on the upper surface of the main board 1400 of this embodiment, a battery socket 1410 may be formed for installing a battery for power supply, and a board controller 1420 may be formed to analyze electrical signals transmitted from the reaction electrodes 130, the reference electrode 140, and the counter electrode 150.

As shown in FIGS. 14 to 16, the upper case 1500 of this embodiment, when combined with the lower case 1200, forms an accommodation space between the upper case 1500 and the lower case 1200 to accommodate the main board 1400, the sensor board 1300, and the microneedle sensor 100. The upper case 1500 and the lower case 1200 may protect the main board 1400, the sensor board 1300, and the microneedle sensor 100 from external shock or foreign substances.

FIG. 17 is a graph showing the blood glucose measurement values (marked with ‘.’) of body fluid measured by a continuous blood glucose meter consisting of the continuous body fluid meter 1000 of this embodiment and the blood glucose measurement values (marked with ‘_’) measured by a conventional blood glucose meter over time.

At this time, a continuous blood glucose meter is attached to the skin and measures in real time changes in glucose in the body fluid, and a blood glucose meter measures blood glucose from actual blood collected at regular intervals.

Here, a continuous blood glucose meter is administered and attached to the skin of a rat, the subject of blood glucose measurement, and then a 20-minute stabilization period is performed. After 20 minutes, a 30% glucose solution (G) (1.0 g/kg body weight) is administered and changes in blood glucose measured by the continuous blood glucose meter are measured over time. Since the measurement results of the continuous blood glucose meter are measured continuously, the measurement results are shown as a solid line (indicated by ‘•’) in the graph in FIG. 17.

In addition, blood is collected from the tail vein of a rat, the subject of blood glucose measurement, every 5 minutes, and then changes in blood glucose values measured by a blood glucose meter are measured. Since the measurement results of the blood glucose meter are measured once every 5 minutes, the measurement results are shown as a plurality of points (marked with ‘▴’) in the graph of FIG. 17.

As shown in FIG. 17, it was confirmed that the solid line of the measurement results measured by the continuous blood glucose meter exhibited a very similar pattern to the dotted line of the measurement results measured by the blood glucose meter. However, while the blood glucose meter measures blood glucose directly from the blood, the continuous blood glucose meter measures the concentration of glucose diffused from the blood into the body fluid, so there is a certain delay time in both measurement results. Accordingly, it can be inferred that it is possible to measure blood glucose using a continuous blood glucose meter if only the blood glucose measurement time equivalent to the delay time may be secured.

According to a microneedle sensor according to an embodiment of the present disclosure, a method of manufacturing the same, and a continuous body fluid meter using the same, since the microneedle sensor has a structure that senses specific substances in body fluid, such as blood glucose, by inserting hollow microneedles into the body's skin while reaction electrodes are inserted into the hollow holes of the hollow microneedles, the microneedle sensor can easily sense the specific substances in body fluid in a minimally invasive manner using the hollow microneedles, and can continuously measure changes in the specific substances in body fluid using changes in electrical signals detected by the reaction electrodes. Therefore, by using the microneedle sensor of this embodiment, a continuous body fluid meter for continuously measuring blood glucose in body fluid over a long period of time can be easily manufactured.

In addition, according to a microneedle sensor according to an embodiment of the present disclosure, a method of manufacturing the same, and a continuous body fluid meter using the same, the microneedle sensor has a structure in which a counter electrode is applied to the second region of a sensor panel and the outer surface of a hollow microneedle placed in the corresponding region and a reference electrode is applied to the first region of the sensor panel and the outer surface of a hollow microneedle placed in the corresponding region. Accordingly, reaction electrodes can be inserted and placed into the hollow holes of all hollow microneedles, and thus the number of reaction electrodes installed on the same number of hollow microneedles can be significantly increased compared to before. Thus, the sensing range and accuracy of the microneedle sensor can be improved.

In addition, according to a microneedle sensor according to an embodiment of the present disclosure, a method of manufacturing the same, and a continuous body fluid meter using the same, since the microneedle sensor has a structure in which only reaction electrodes are placed in the hollow holes of hollow microneedles, unlike the conventional case, hollow microneedles for inserting a counter electrode and a reference electrode are not required. Accordingly, the number of hollow microneedles needed to sense body fluid can be further reduced, and the microneedle sensor can be manufactured compactly to achieve miniaturization and cost reduction of the microneedle sensor.

In addition, according to a microneedle sensor according to an embodiment of the present disclosure, a method of manufacturing the same, and a continuous body fluid meter using the same, by forming a fine roughness plating layer on the surface of a reaction electrode, the sensing sensitivity of the reaction electrode can be improved by increasing the reaction surface area of the reaction electrode. In addition, by changing the length of the hollow hole of a hollow microneedle into which the reaction electrode is inserted, the sensing sensitivity of the reaction electrode can be controlled by adjusting the surface area of the reaction electrode in contact with body fluid.

As described above, in the present disclosure, specific matters such as specific components are explained by limited examples and drawings, but these are provided to facilitate an overall understanding of the present disclosure, and the present disclosure is not limited to the embodiments. Those skilled in the art will appreciate that various modifications, additions, and substitutions are possible, without departing from the scope and spirit of the invention. Accordingly, the spirit of the present disclosure is not limited to the described embodiments. In addition to the claims described later, all equivalent or equivalent variations of these claims fall within the scope of the present disclosure.

What is claimed is:

1. A microneedle sensor, comprising:
a sensor panel placed so that one surface thereof is in contact with skin of a body;
a plurality of hollow microneedles that are formed on one surface of the sensor panel to protrude in form of micrometer-scale needles, wherein a hollow hole with one side exposed to outside is provided inside the hollow microneedle;
reaction electrodes of a conductive material each inserted into the hollow holes of the hollow microneedles and configured to be inserted into an interior of the skin along with the hollow microneedles;
a reference electrode of a conductive material applied to a first region formed on one surface of the sensor panel and applied to outer surfaces of hollow microneedles located on the first region; and
a counter electrode of a conductive material applied to a second region formed on one surface of the sensor panel and applied to outer surfaces of hollow microneedles located on the second region.

2. The microneedle sensor according to claim 1, wherein the first region and the second region are provided at positions spaced apart from each other on one surface of the sensor panel, and
the hollow microneedles are disposed in both the first and second regions.

3. A microneedle sensor, comprising:
a sensor panel placed so that one surface thereof is in contact with skin of a body;
a plurality of hollow microneedles that are formed on one surface of the sensor panel to protrude in form of micrometer-scale needles, wherein a hollow hole with one side exposed to outside is provided inside the hollow microneedle;
reaction electrodes of a conductive material inserted into hollow holes of some of the hollow microneedles and configured to be inserted into an interior of the skin along with the hollow microneedles;
a reference electrode of a conductive material inserted into a hollow hole of a remaining one of the hollow microneedles; and
a counter electrode of a conductive material applied to one surface of the sensor panel and applied to outer surfaces of the hollow microneedles.

4. A microneedle sensor, comprising:
a sensor panel placed so that one surface thereof is in contact with skin of a body;
a plurality of hollow microneedles that are formed on one surface of the sensor panel to protrude in form of micrometer-scale needles, wherein a hollow hole with one side exposed to outside is provided inside the hollow microneedle;
reaction electrodes of a conductive material each inserted into the hollow holes of the hollow microneedles and configured to be inserted into an interior of the skin along with the hollow microneedles; and
a reference electrode of a conductive material applied to one surface of the sensor panel and applied to outer surfaces of the hollow microneedles.

5. The microneedle sensor according to claim 1, wherein, on one side of the hollow microneedle, an opening is formed by cutting the one side of the hollow microneedle, and
the one side of the hollow hole is exposed to outside through the opening.

6. The microneedle sensor according to claim 5, wherein the hollow hole is configured to extend long from an inside of the hollow microneedle to penetrate the sensor panel, and
the reaction electrodes are formed as wires that penetrate the sensor panel and are inserted into the hollow holes.

7. The microneedle sensor according to claim 5, wherein one end of the reaction electrode is movably inserted into the hollow hole, and
the reaction electrode controls sensing sensitivity of body fluid by adjusting a length of the hollow hole and changing a reaction area exposed through the opening.

8. The microneedle sensor according to claim 5, wherein the opening is formed in various sizes on one side of the hollow microneedle, and
the reaction electrode controls sensing sensitivity of body fluid by changing a reaction area exposed through the opening depending on a size of the opening.

9. The microneedle sensor according to claim 1, wherein the reaction electrode comprises electrode wire of a conductive material with a fine roughness plating layer formed on a surface thereof;
a sensor layer that is applied to one end of the electrode wire inserted into the hollow hole and comprises a substance that reacts to specific sensing substances in body fluid; and
a diffusion control layer applied to one end of the electrode wire to surround the sensor layer and configured to restrict passage of interfering substances within the body fluid that interferes with reaction with the specific sensing substances.

10. The microneedle sensor according to claim 9, wherein the reaction electrode further comprises a biocompatible membrane that is applied to surround the sensor layer and prevents biofouling due to an immune response when the hollow microneedles are inserted into skin; and
an insulating layer that is applied to the other end of the electrode wire where the biocompatible membrane is not formed and insulates the electrode wire.

11. The microneedle sensor according to claim 9, wherein the sensor layer and the diffusion control layer are applied by dip coating to one end of the electrode wire.

12. The microneedle sensor according to claim 9, wherein the sensor layer comprises glucose oxidase (GOx) and an electron transfer mediator (osmium redox polymer) to measure blood glucose in the body fluid, and
the diffusion control layer comprises Nafion.

13. The microneedle sensor according to claim 9, wherein, on a surface of the electrode wire, a fine roughness plating layer with micropores is formed depending on electrodeposition of Pt black (platinum powder).

14. The microneedle sensor according to claim 1, wherein the reaction electrodes are formed of any one of gold (Au), platinum (Pt), silver (Ag), carbon (C), copper (Cu), palladium (Pd), and stainless steel,
the reference electrode is formed of any one of standard hydrogen electrode (SHE), calomel (Hg/Hg₂Cl₂), and silver-silver chloride (Ag/AgCl), and
the counter electrode is formed of any one of gold (Au), platinum (Pt), silver (Ag), carbon (C), copper (Cu), palladium (Pd), and stainless steel.

15. The microneedle sensor according to claim 4, wherein the reaction electrodes are formed of any one of gold (Au), platinum (Pt), silver (Ag), carbon (C), copper (Cu), palladium (Pd), and stainless steel, and
the reference electrode is formed of any one of standard hydrogen electrode (SHE), calomel (Hg/Hg₂Cl₂), and silver-silver chloride (Ag/AgCl).

16. A continuous body fluid meter, comprising the microneedle sensor according to claim 1.

17. The continuous body fluid meter according to claim 16, further comprising:
a lower case having a sensor hole where the microneedle sensor is placed so that the hollow microneedles of the microneedle sensor are exposed to outside;
a sensor board that is combined with the microneedle sensor to receive electrical signals from the microneedle sensor;
a main board that is connected to the sensor board and continuously measures specific sensing substances in body fluid using electrical signals of the microneedle sensor; and
an upper case coupled to the lower case to accommodate the main board, the sensor board, and the microneedle sensor therein.
